(19) United States
(12) Patent Application Publication
(10) Pub. No.: US 2015/0037832 A1
(43) Pub. Date: Feb. 5, 2015

(54) METHODS OF FACILITATING THE BIOCONVERSION OF CRUDE BIODIESEL-DERIVED GLYCEROL BY MICROORGANISMS

(71) Applicants: Carmen Scholz, Madison, AL (US); Keerthi Prasad Venkataramanan, Bear, DE (US); Geoffrey D. Bothun, West Kingston, RI (US)

(72) Inventors: Carmen Scholz, Madison, AL (US); Keerthi Prasad Venkataramanan, Bear, DE (US); Geoffrey D. Bothun, West Kingston, RI (US)

(21) Appl. No.: 14/342,515

(22) PCT Filed: Sep. 1, 2012
(86) PCT No.: PCT/US2012/053563

§ 371 Date: Sep. 11, 2014

Related U.S. Application Data
(60) Provisional application No. 61/530,250, filed on Sep. 1, 2011.

(51) Int. Cl. C12P 7/16; C07C 29/76

(52) U.S. Cl. CPC C12P 7/16; C07C 29/76
USPC 435/29; 435/160

(57) ABSTRACT

The present disclosure generally pertains to a method of facilitating the bioconversion of glycerol by microorganisms. In one embodiment, biodiesel derived crude glycerol is purified by removing fatty acids through acid precipitation. The fatty acid-free crude glycerol is then utilized as a carbon source for the culture of microorganisms and the production of value added substances. Additionally, the unsaturated fatty acids within the biodiesel-derived crude glycerol are converted to saturated fatty acids, allowing for fermentation behavior similar to that of pure glycerol. Both the cultured microorganisms and the culture media containing the purified crude glycerol may be analyzed for crude glycerol bioconversion products. The disclosure also relates to a method of increasing product yield through the addition of a second carbon source to the microorganism culture media.

### CROSS REFERENCE TO RELATED APPLICATION

This is the national stage application of and claims priority to International Application No. PCT/US2012/053563, entitled “Methods of Facilitating the Bioconversion of Crude Biodiesel-Derived Glycerol by Microorganisms” and having an international filing date of Sep. 1, 2012, which is incorporated herein by reference. International Application No. PCT/US2012/053563 claims priority to U.S. Provisional Patent Application No. 61/530,250, entitled “Butanol Production by Clostridium pasteurianum ATCC 6013 Using Biodiseal-Derived Crude Glycerol: Microbial Response to Environmental Stress” filed on Sep. 1, 2011, which is incorporated herein by reference.

### STATEMENT REGARDING FEDERALLY SPONSORED RESEARCH OR DEVELOPMENT

This invention was made with Government support under NSF, CBET-0966846 awarded by the National Science Foundation. The Government has certain rights in the invention.

### RELATED ART

The biofuel industry has experienced tremendous growth in response to increasing environmental concerns and the need for alternate fuel sources, resulting in the production of over 315 million gallons of biodiesel in 2010. The production of biodiesel employs transesterification of plant oils or animal fats, containing triacylglycerol, with methanol in the presence of a strong base. This process produces a glycerol co-product at 10% (w/w) containing impurities, such as methanol, ash/salts, and residual fatty acids, often referred to as “crude” glycerol. The rapidly expanding production of biodiesel is therefore dramatically altering the cost and availability of glycerol. The crude glycerol generated during biodiesel production has very limited usability due to the presence of impurities. As a result, crude glycerol is currently considered a waste product and has little commercial value. Conventional uses of glycerol, such as in cosmetics and pharmaceuticals, are not possible with crude glycerol because of the need for a highly pure product. The purification of crude glycerol is an expensive process, providing little financial incentive to purify the by-product. One alternative for utilizing this glycerol by-product is its biological conversion into value-added products in the form of solvents and organic acids, such as butanol, 1,3-propanediol (PDO), citric acid, omega-3 polyunsaturated fatty acid and succinic acid.

Microorganisms such as Clostridium pasteurianum ATCC 6013 are capable of fermenting crude glycerol and converting it to a variety of chemical compounds. The most notable compound is butanol, considered a key chemical component for the production of bio-based alternatives to petroleum chemicals. The production of butanol from glycerol using C. pasteurianum is therefor of particular interest. However, the fermentation of crude glycerol presents difficulties in terms of reproducibility, product yield and significant variations in fermentation duration. These problems result from the variability in crude glycerol composition and the fact that impurities inhibit microbial activity and fermentation. Bacterial growth rates are slower and product yields are lower on crude glycerol in comparison to the results observed with pure glycerol. In addition, the resulting butanol produced by the bacteria becomes toxic to the cells at concentrations of 2%.

Although certain microorganisms are capable of utilizing biodiesel-derived crude glycerol for the production of certain value-added products, the fermentation processes involved have not been optimized to maximize product yield. It has been shown that the product distribution during fermentation can be altered by modifying microorganism growth conditions, for instance, media composition and pH. One possible modification includes incorporating additional carbon sources, such as sugars. Examining the utilization of sugars could result in the use of these molecules from renewable sources for the co-fermentation with glycerol.

As a result, there is a need for a method to remove impurities from crude glycerol samples to provide a suitable carbon source in order to increase C. pasteurianum growth, metabolism and production of value-added crude glycerol products. In addition, there is a need for a method to enhance glycerol utilization and improve the yield of value-added products.

### BRIEF DESCRIPTION OF THE DRAWINGS

The disclosure can be better understood with reference to at least the following drawings. The elements of the drawings are not necessarily to scale relative to each other, emphasis instead being placed upon clearly illustrating the principles of the disclosure.

FIG. 1 illustrates the degree of unsaturation of fatty acids present in exogenous butanol added to media containing glucose and the endogenously produced butanol from media containing glycerol.

FIG. 2A is a graphical comparison of metabolism during glycerol-glucose fermentation illustrating a growth profile.

FIG. 2B is a graphical comparison of metabolism during glycerol-glucose fermentation illustrating a pH profile.

FIG. 3A is a graphical comparison of metabolism during glycerol and L-arabinose co-fermentation illustrating a growth profile.

FIG. 3B is a graphical comparison of metabolism during glycerol and L-arabinose co-fermentation illustrating a pH profile.

FIG. 4A is a graphical comparison of metabolism during glycerol and D-xylose co-fermentation illustrating a growth profile.

FIG. 4B is a graphical comparison of metabolism during glycerol and D-xylose co-fermentation illustrating a pH profile.

FIG. 5A is a graphical illustration of the effect of the addition of acetate, butyrate and biotin on glycerol fermentation in terms of a growth profile.

FIG. 5B is a graphical illustration of the effect of the addition of acetate, butyrate and biotin on glycerol fermentation in terms of a pH profile.

### DETAILED DESCRIPTION

The present disclosure pertains to a method of facilitating the bioconversion of crude glycerol obtained during the production of biodiesel fuels by bacteria to produce value-added products. In one embodiment, a method comprises the removal of fatty acid impurities from biodiesel derived crude glycerol samples comprising (1) adjusting the pH of the sample to approximately 2.0-3.0 so that the fatty acids precipitate from solution and (2) removing the precipitated fatty acids from the sample. The resulting purified glycerol is then an appropriate substrate for bacterial growth and the production of value added products, for example butanol.

The term “biodiesel” is defined as the methyl esters of acyl fatty acids. The production process of biodiesel therefor includes the transesterification of vegetable oils or animal fats, containing triacyl glycerols, with methanol in the presence of a strong base to form biodiesel and the co-product glycerol. This glycerol, herein defined as “biodiesel-derived crude glycerol” or simply “crude glycerol” contains numerous impurities, including residual fatty acids, thus limiting its use. The disclosure further relates to the removal of fatty acids from the biodiesel derived crude glycerol samples.

The fermentation of glycerol has been observed in some pathogens such as Citrobacter and Klebsiella and in some species of algae. However, the majority of pathogens do not have the ability to use glycerol as the sole carbon source. The disclosure therefor contemplates a microorganism capable of utilizing glycerol as a carbon source. Clostridium pasteurianum belongs to a genus of Gram-positive, rod shaped bacteria These organisms are obligate anaerobes capable of producing endospores. C. pasteurianum is of particular interest in this disclosure due to its non-pathogenicity, its ability to tolerate high concentrations of glycerol and it ability to produce a wide range of products using crude glycerol as the sole carbon source. C. pasteurianum has been shown to grow in the presence of crude glycerol while yielding butanol, ethanol, 1,3-propanediol and butyrate as a metabolic by-products. Butanol is a product of considerable interest in this disclosure due to its high energy content (as compared to ethanol), its miscibility with petroleum products and its low vapor pressure.

The metabolism of crude glycerol is much slower than that of pure glycerol. In addition, the amount of time needed for complete substrate utilization is much slower for crude glycerol as compared to pure glycerol. The delay in the utilization of crude glycerol is attributed to the presence of impurities. The disclosure further describes the effects of impurities in crude glycerol on C. pasteurianum and shows fatty acids to be the major inhibitors of metabolism, growth and butanol production. The inventors have surprisingly discovered that other impurities, such as salt and alcohol, have a negligible effect on microorganism fermentation processes. Instead, the inventors have found that the presence of unsaturated fatty acids in crude glycerol imped its fermentation by microorganisms and its conversion into the value-added material butanol. For the purpose of this disclosure the term “fatty acid” refers to any residual fatty acids remaining in the crude glycerol sample after esterification. In one aspect, therefore, the disclosure relates to methods of eliminating impurities from biodiesel derived crude glycerol to enhance its utilization and value-added conversion process.

In an additional aspect, the disclosure relates to a method of removing fatty acids from bio-derived crude glycerol samples comprising lowering the pH of the sample to between 2.0 and 3.0. In one embodiment, an appropriate amount of a strong acid, for example hydrochloric acid (HCl), is added to the sample to lower the pH to within this range. As used herein, an appropriate amount is the amount of acid for adjusting the pH of the sample to the proper range. The appropriate amount of acid required will depend on several factors including the identity and concentration of the acid, the concentration of the crude glycerol sample, the concentration impurities present in the crude glycerol and the volume of the sample. Other strong acids may be used as is known to one of skill in the art, for example HNO₃ (nitric acid), H₂SO₄ (sulfuric acid), HBr (hydrobromic acid) and HI (hydroiodic acid—also known as hydriodic acid). At this pH range fatty acids will precipitate from the crude glycerol solution. The fatty acids are then removed from the sample. In one exemplary embodiment, the precipitated fatty acids are removed by centrifugation. However, other methods of separating the precipitated fatty acids may be employed, for instance chromatography (liquid or supercritical fluid chromatography), chemical or thermal fractionation, solvent extraction or distillation. Removal of the fatty acids from the crude glycerol results in a purified glycerol sample containing only trace amounts of fatty acids.

The purified glycerol is then added to a microbial growth media where it will be utilized as a carbon source. In one aspect of the disclosure, the purified glycerol is used to supplement a growth media acceptable for the growth of prokaryotic organisms, for instance bacteria. The growth medium or culture medium may be a liquid or gel designed to support the growth of microorganisms or cells. It is to be understood that the purified glycerol is acceptable for use as a carbon source in other types of media, for instance media for the growth of eukaryotic cells such as algae. The growth media may be a liquid or mixed with agar. Examples of bacterial growth media which can utilize the purified glycerol as described above is nutrient rich reinforced clostridal growth media (RCM), defined media and Biebl media the components of each are described in detail below. It is to be understood that the use of RCM or defined media is purely exemplary and other suitable media may be used in the disclosure as would be known to one of skill in the art. The growth media containing the purified glycerol is herein after referred to as purified glycerol growth media.

Economic considerations are always important factors in developing cell culture procedures. The disclosure therefore contemplates methods for enhanced substrate utilization and improved product yield. In one aspect, the inventors have found that modification of the growth conditions can change the microbial fermentation characteristics. For instance, the incorporation of a second carbon source (in addition to glycerol) into bacterial culture media increased the production of value-added products. The carbon flow from glycerol into various value-added products can be regulated by making appropriate additions to the media. Therefore, an object of the disclosure relates to the addition of a secondary carbon source to the purified glycerol growth media. In one embodiment, the second carbon source is a sugar. For instance, the inventors have found that the addition of glucose, as a co-substrate of glycerol, resulted in an increased production of butanol. The butanol production doubled with the addition of glucose at half the concentration of glycerol. Co-fermentation of glycerol with glucose resulted in a maximum increase of butanol formation of 110%. The disclosure therefore embodies the addition of glucose to the purified glycerol growth media as a secondary carbon source. It is to be understood that the methods of the disclosure are not restricted solely to the addition of glucose, or sugars in general, and that other carbon sources may be utilized to increase product formation.

In an additional embodiment of the disclosure, the purified glycerol growth media may be used to support the growth of the microorganisms or cells of choice. The microorganisms or cells may be introduced to the media (inoculated) by various means as is known in the art. Preferably, the microorganisms or cells are able to utilize glycerol as a carbon source. In an additional embodiment, the microorganisms or cells are able to utilize glycerol as the sole carbon source. In one aspect, bacterial cells are inoculated into the purified glycerol growth media. Once such exemplary bacterial cell is Clostridium pasteuruanum. The cells are then grown at the appropriate conditions, i.e., optimal O₂, CO₂, N₂ and H₂ concentration, temperature and humidity. The conditions required for optimal growth of the cells are dependent on the cell type and the identity of the specific value added product produced.

In another embodiment, the cells are permitted to grow for a specified period of time after which the cells, the purified glycerol growth media, or both the cells and growth media are analyzed for the presence of produced value-added products. For instance, samples of growth media or cells may be obtained from the cell culture on a periodic basis during cell culture. In an exemplary embodiment, culture media may be tested for the presence and concentration of glycerol, acetic acid, 1,3-propanediol, ethanol, butyric acid and butanol. In the present disclosure, samples may be analyzed by a number of methods as known in the art, for example, High Performance Liquid Chromatography (HPLC), Nuclear Magnetic Resonance spectroscopy (NMR), chromatography, etc. Cell samples may be tested and analyzed utilizing methods known in the art.

In an additional embodiment, the present disclosure illustrates that of all the impurities present in crude glycerol, only fatty acids with one or more double bonds are responsible for impeding microbial growth. The inventors have discovered that removal of fatty acids, particularly unsaturated fatty acids, from crude glycerol samples results in increased metabolic and growth rates of C. pasteurianum when utilizing the crude glycerol as the carbon source. In addition, removal of fatty acids increased the rate and overall production of butanol. Removing fatty acids that contain double bonds renders the crude glycerol fermentable by a microorganism, for example, C. pasteurianum. The inventors have found that microbial growth behavior and the production of butanol with glycerol free of fatty acids are identical to the growth and behavior observed with pure glycerol.

In an additional embodiment, therefore, the disclosure further relates to the removal of unsaturated fatty acids, i.e., those containing one or more double bonds in their alkyl tail, from the crude glycerol. In one exemplary embodiment, an unsaturated fatty acid may be converted to a saturated fatty acid by a hydrogenation reaction. The process of hydrogenation adds hydrogen atoms to unsaturated fatty acids, eliminating double bonds and making it into a partially or completely saturated fatty acid. The addition of hydrogen to an alkene (an unsaturated molecule) results in the production of an alkane (a saturated molecule):

H₂C═CH₂+H₂-->CH₃CH₃

In one embodiment, hydrogen gas is bubbled through the crude glycerol sample in the presence of a catalyst, e.g., a reactive metal. Hydrogenation of the crude glycerol will saturate double bonds present in the fatty acids, thus rendering the substrate fermentable. It will be recognized that other methods of removing the unsaturated portions of the fatty acid are available as is known in the art. Glycerol contains no other unsaturated components. As a result, no other adverse reactions in the crude glycerol will occur.

In order to promote a further understanding of the invention, the following examples are provided. These examples are shown by way of illustration and not limitation.

### Example 1

### Butanol Production and Effect of Impurities

### Experimental

### Materials

Biodiesel-derived crude glycerol was obtained from the Green River Biodiesel, Moundville, Ala. All chemicals were purchased from either Fischer Scientific or Sigma-Aldrich, unless mentioned otherwise. Fatty acids such as sodium stearate, sodium oleate and sodium linoleate were obtained from TCI America.

Bacterial Strain, Media and Fermentation

Clostridium pasteurianum ATCC 6013 was obtained from American Type Culture Collection (Manassas, Va.). Freeze dried pure cultures of C. pasteurianum were revived using nutrient rich reinforced clostridal growth media (RCM) and glycerol stock cultures were prepared for future use. The RCM media was obtained from Becton, Dickenson and Company. Pre-culture for glycerol utilization experiments were grown in RCM. The growth was monitored using optical density and the cultures were used as inoculum once they reached mid-exponential phase of growth. The growth experiments were conducted in 75 mL culture flasks containing 40 mL of the media and 5 mL of inoculum. Defined media was used for glycerol utilization. Unless otherwise specified purified glycerol or biodiesel derived crude glycerol were used as the sole carbon source. All experiments were conducted in an anaerobic chamber (95% N₂ and 5% H₂) at a temperature of 37° C. to ensure oxygen-free conditions. The pH of the samples were measured at regular intervals and the samples was analyzed for glycerol, acetic acid, 1,3-propanediol, ethanol, butyric acid and butanol by High Performance Liquid Chromatography (HPLC) equipped with a refractive index detector and a Bio-Rad Aminex 87-H column using 5 mM sulfuric acid as the mobile phase.

Effect of Impurities

To study the effect of impurities in crude glycerol, various concentrations of each impurity were added to the media containing 25 g/L pure glycerol. Methanol was added at 2.5 g/L and 5 g/L. Potassium chloride and potassium sulfate were added at 2.5, 5, 7.5 and 10% (w/w) of the initial glycerol concentration. The fatty acids were added in the form of sodium salts of steric acid, oleic acid and linoleic acid at 5, 10 and 15% (w/w) (of initial substrate conc.). The media containing 25 g/L of pure glycerol was used as the control. The effects of fatty acids were also studied by partially purifying the crude glycerol by precipitating the fatty acids using concentrated hydrochloric acid. The crude glycerol was diluted in deionized water at a ratio of 1:3 and hydrochloric acid was added to lower pH to between 2 and 3. At this pH the fatty acids precipitated and were removed by centrifugation at 5000 rpm for 15 minutes. The precipitated fatty acids were later analyzed using NMR by dissolving them in d-DMSO.

Effect of Butanol

The effect of butanol on growth of the bacteria and the stability and change in composition of the membrane were studied by adding butanol to the media containing wither glucose or glycerol as the sole carbon source. The glucose was used to analyze the effect of exogenous butanol, as C. pasteurianum does not produce butanol when growing on glucose. The effect of endogenously produced butanol was studied by varying the glycerol concentration from 10 g/L to 50 g/l. The concentration of butanol added to the media varied from 0 to 2%. The cells were allowed to grow in the presence of butanol for 24 hours after which the membrane was extracted.

Extraction of Cell Membrane

The cell membrane was extracted using dichloromethane/methanol mixtures. The membrane extracted in dichloromethane was separated by evaporating the solvents under a gentle nitrogen stream. This dry film of the membrane was used for further analysis.

Analysis of the Membrane

The dry film of the membrane was dissolved in deuterated chloroform for NMR analysis. Synthetic lipids such as Diaplmitoylphosphotidylcholine (DPPC) and Dioleylphosphotidylcholine (DOPC) were used as standards for NMR analysis. The dry films of the membrane lipids were labeled with DPH (500:1) and made into liposomes for analyzing the stability of the membrane by studying the fluorescence anisotropy using a Perkin-Elmer LS50B.

### Results and Discussion

Effect of Impurities

The effect of methanol was studied at concentrations of 2.5 g/l and 5 g/L. These concentrations were selected based on the information from the material safety data sheet obtained for crude glycerol. At these concentrations of methanol, no reduction in growth or product formation during the fermentation of glycerol was observed. Potassium chloride and potassium sulfate had no detrimental effect on growth and product yield.

The effect of fatty acids was studied using three different fatty acids: sodium stearate, sodium oleate and sodium linoleate. These three fatty acids differed in the degree of unsaturation from 0, 1 and 2 double bonds, respectively, and had the same chain length (C₁₈). In presence of sodium stearate and sodium oleate, the substrate utilization was not affected at all three concentrations (5%, 10% and 15%) tested. These concentrations were chosen based on the amount of fatty acids determined in crude glycerol, which was found to be 11.65±1.8% (n=3). The fermentation in Clostridia involves biphasic metabolism of acidogenesis and solventogenesis, and this biphasic metabolism was observed in the presence of sodium stearate and sodium oleate. In the presence of sodium linoleate, which has a higher degree of unsaturation compared to the other two fatty acids that were tested, substrate utilization was completely inhibited at all three concentrations. This shows an increasingly inhibitive effect of fatty acids with an increase in the degree of unsaturation towards the utilization of glycerol in C. pasteurianum. The separation of fatty acids in crude glycerol using acid precipitation resulted in a better utilization of the partially purified crude glycerol and the yields matched that were obtained from pure glycerol.

Effect of Butanol on Cell Membrane

The effect of exogenous butanol was studied by adding butanol to mid-log cultures of C. pasteurianum growing in the defined media containing 50 g/L of glucose as the substrate and also in RCM. These two media were chosen because butanol is not produced by C. pasteurianum growing on glucose. This experimental setup provides an ideal platform to study the effect of exogenous butanol added to the media.

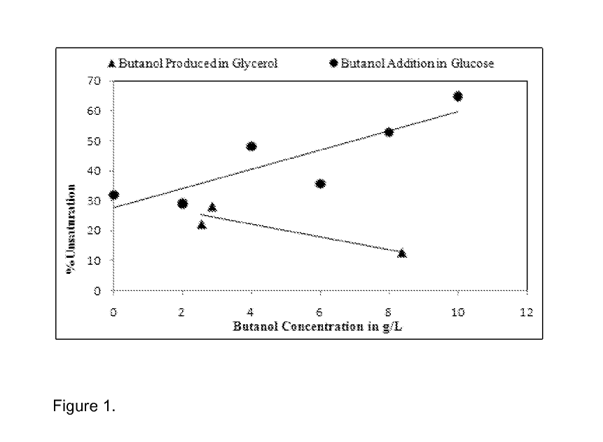

Butanol was added at various concentrations from 2 g/L to 20 g/L and isolated membranes were analyzed for stability by measuring anisotropy and degree of unsaturation through NMR. The proton signal corresponding to the alkenes in the fatty acid side chain was observed at 5.29 ppm which was verified using DOPC as the standard lipid. The percentage of unsaturation in each sample was calculated using the standard curve obtained from using mixtures of DPPC and DOPC (0-100, 25-75, 50-50, 75-25, 100-0). In both the defined media containing glucose and the RCM, the degree of unsaturation was observed to increase with the increase in the amount of exogenous butanol concentrations. The anisotropies of the samples were found to decrease with the increase in butanol concentration showing an increase in the fluidity of the membranes, which correlated to the presence of butanol in the media (FIG. 1).

The effect of endogenously produced butanol was studied by using the defined media and varying the glycerol concentration which resulted in the production of different concentrations of butanol. The NMR analysis revealed a decrease in the degree of unsaturation (FIG. 1) along with a corresponding increase in anisotropy. This result shows that during endogenous butanol production the bacteria lower the degree of unsaturation in the membrane to tolerate more butanol that it produces, which is supported by the anisotropy data. That is, there is a decrease in fluidity and the membranes become more rigid.

### Conclusions

The analysis of the effect of impurities present in crude glycerol shows that the fatty acids are the major inhibitors of substrate utilization, hence, growth and metabolism of C. pasteurianum. The removal of the fatty acid is an important step to ensure utilization of the crude glycerol.

The study on the effect of butanol on the cell membrane shows that the bacteria reduce the degree of unsaturation in their membrane in response to the amount of butanol that they produce during glycerol formation. From these results one can conclude that the mechanism of butanol production and butanol tolerance (reduction in the unsaturation of the fatty acid tail) seem to be interrelated as the bacterial membrane is susceptible to the increase in fluidity due to exogenous butanol, when butanol is added in non-butanol production media.

### Example 2

### Supplementation with a Secondary Carbon Source

### Experimental

### Materials and Methods

Clostridium pasteurianum ATCC™ 6013 was obtained from American Type Culture Collection (Manassas, Va.). Freeze dried pure cultures of C. pasteurianum were revived using nutrient-rich reinforced clostridial growth media (RCM) and glycerol stock cultures were prepared as above.

Pre-cultures for glycerol utilization experiments were grown in RCM. The growth was monitored using optical density (λ=600 nm) and the cultures were used as inoculum once they reached the mid-exponential phase of growth. 100 mL of Biebl medium with appropriate carbon sources was inoculated with 10% inoculum and the contents were split equally into three in a 75 mL culture flask. All experiments were carried out in batch cultures and in triplicates. The cultures were monitored for growth, substrate utilization and product formation as mentioned earlier.

The co-fermentation of sugars was studied using hexose (dextrose) and pentose (D-xylose and L-arabinose) with 20 g/L of pure glycerol at a concentration of 10 g/L of the sugars, maintaining a 2:1 ratio of glycerol and sugars. Cells grown on glycerol or sugar alone were used as controls. Acetate and butyrate, the acid co-product in butanol pathways, were studied for their impact on butanol production by supplementing the media with sodium acetate and sodium butyrate, respectively. Acetate and butyrate were studied at concentrations of 2 g/L with an initial glycerol concentration of 25 g/L of pure glycerol. Biotin was supplemented in the media at 0.8 mg/L with an initial concentration of 25 g/L of pure glycerol. A stock solution of 400 mg/L of biotin was made in 95% ethanol with sterile, autoclaved deionised water and an appropriate amount was added to the media, after the media were sterilized.

All these experiments were conducted using pure glycerol as the use of partially purified crude glycerol resulted in the lower yields of 1,3-PDO.

### Results and Discussion

Cofermentation of Glycerol with Sugars

The co-fermentation of glycerol was studied with glucose, arabinose and xylose. Controls for the experiments were cultures grown on each carbon as the sole carbon source. The cultures on the control experiments were grown until little or no carbon source was left behind, as analyzed using HPLC.

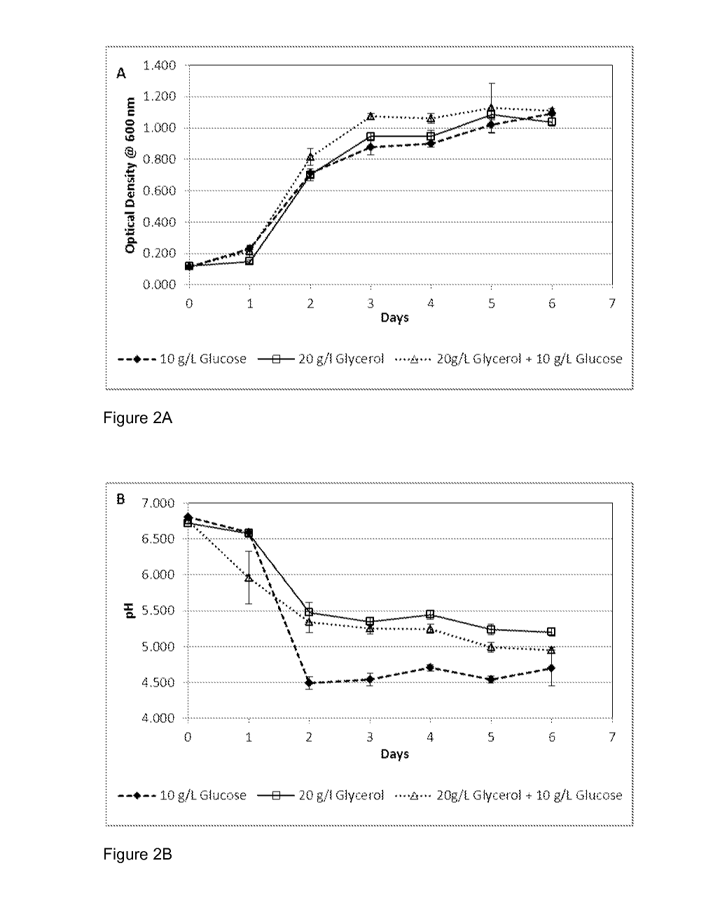

FIG. 2 to FIG. 5 compare the optical densities (measured at 600 nm) and pH profiles of the cultures grown at various conditions. The optical densities of the control as well as the cultures grown under co-fermentation followed the S-shaped (sigmoidal) growth curve typically observed in bacteria. FIGS. 2A and 2B compare the metabolism during glycerol-glucose co-fermentation. FIG. 2A compares the growth profile of cultures in glucose, glycerol and glycerol & glucose. The growth curves of cultures grown in all three conditions are similar. A comparison using the maximum specific growth rate, μₘₐₓ, calculated during the maximum growth rate in the exponential log phase, shows that the cultures in co-metabolism of glycerol and glucose grew at a faster rate than the two controls. The μₘₐₓ for cultures on glycerol were calculated as 1.562±0.118 day⁻¹, while the cultures on glucose and glycerol-glucose had a μₘₐₓ of 1.130±0.055 day⁻¹ and 1.355±0.115 day⁻¹, respectively. Though the glycerol-glucose cultures had a μₘₐₓ slightly lower than the cultures grown only on glycerol and glucose, respectively, the three cultures were able to grow to the same maximum cell density. The overlapping curves of the three cultures also indicate the absence of a diauxic growth, usually observed in bacteria grown on two carbon sources. During diauxic growth, the bacteria utilize one carbon source first and reach a lag phase, which is followed by another exponential growth phase resulting from the utilization of the second carbon source. The absence of diauxic growth also indicates that glycerol and glucose were simultaneously consumed for growth and metabolism by C. pasteurianum.

Similarly, comparison of the pH profile (FIG. 2B) of the cultures supports the growth data. The cultures grown on glucose reached a highly acidic pH of 4.5, which can be explained by the production of acids (Table 1). The pH profile of the cells on glycerol-glucose co-fermentation closely aligns with the pH profile of the cells on glycerol only. The initial difference in the glycerol-glucose cultures can be described due to the simultaneous consumption of both the carbon sources.

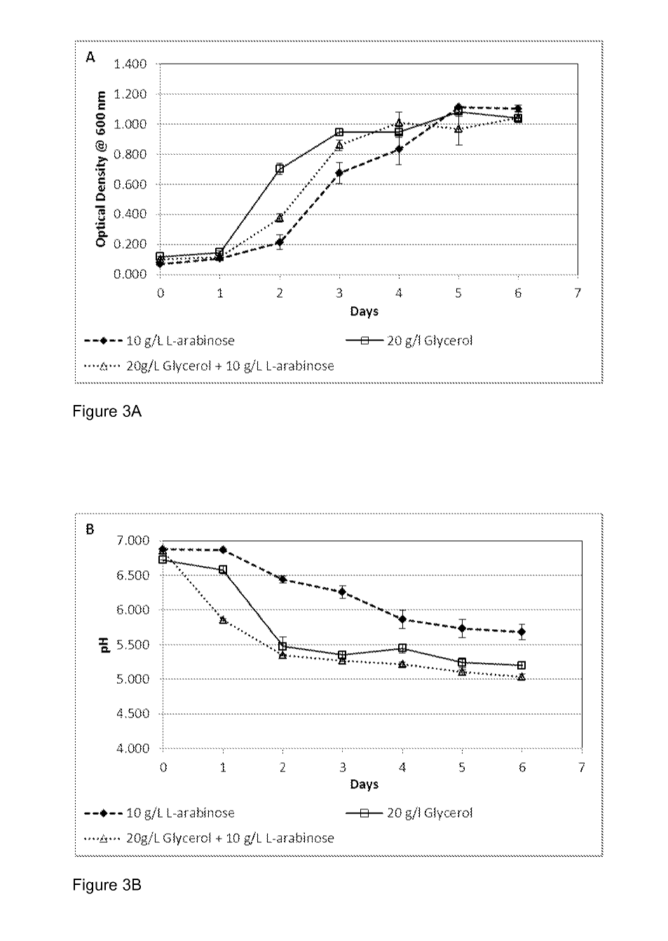

FIGS. 3A and 3B compare the growth and pH profiles of C. pasteurianum cells on the co-metabolism of glycerol and L-arabinose along with the controls. The growth profiles clearly indicate that the glycerol control had a superior growth rate in comparison to the cultures on dual carbon sources and the control on L-arabinose. The μₘₐₓ of the cultures on glycerol and L-arabinose mixture were found to be 1.005±0.020 day⁻¹, and the cultures on arabinose had a μₘₐₓ of 0.933±0.054 day⁻¹.

The comparison of the pH profiles (FIG. 3B) indicates that the controls grown on L-arabinose had the least change to their pH due to the very low production of acids (Table 1), while the cultures on dual carbon source (glycerol and L-arabinose) had the maximum drop in the pH to 5.0 (FIG. 3B). The analysis of growth and pH profile does not show any diauxic growth during co-fermentation of glycerol and L-arabinose.

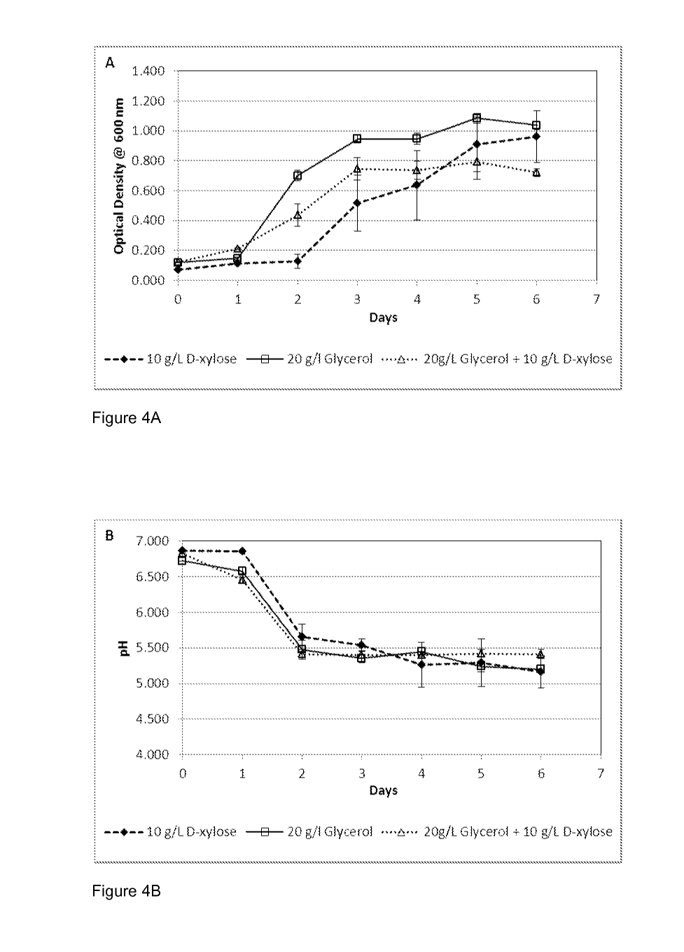

FIGS. 4A and 4B compare the growth and metabolism of C. pasteurianum during D-xylose addition to glycerol with its respective controls. The growth of cells in D-xylose or in the combination of glycerol and D-xylose was very slow and had a very low growth rate. The cultures on D-xylose had a μₘₐₓ of 0.794±0.390 day⁻¹ while the addition of D-xylose to glycerol had the lowest μₘₐₓ of 0.632±0.050 day⁻¹. The pH profile of all the cultures (FIG. 3.3B) have a similar profile. Unlike the co-fermentation of glycerol with glucose and L-arabinose respectively, the co-fermentation of glycerol with xylose resulted in the utilization of glycerol only while more than 90% of D-xylose was left unconsumed in the media.

TABLE 1
Control Experiments - Product yields g/g (mol/mol).

| | Acetate | 1,3-PDO | Ethanol | Butyrate | Butanol |
|---|---|---|---|---|---|
| 10 g/L | 0.21 ± 0.01 | 0.02 ± 0.0 | 0.02 ± 0.0 | 0.41 ± 0.01 | 0.02 ± 0.0 |
| Glucose | (0.64 ± 0.02) | (0.04 ± 0.0) | (0.07 ± 0.0) | (0.83 ± 0.02) | (0.04 ± 0.00) |
| 10 g/L | 0.06 ± 0.01 | 0.28 ± 0.07 | 0.17 ± 0.03 | 0.01 ± 0.0 | 0.005 ± 0.008 |
| L-arabinose | (0.15 ± 0.02) | (0.55 ± 0.02) | (0.57 ± 0.11) | (0.02 ± 0.0) | (0.01 ± 0.02) |
| 10 g/L | 0.25 ± 0.08 | 0.22 ± 0.04 | 0.22 ± 0.04 | 0.02 ± 0.01 | 0.07 ± 0.05 |
| D-xylose | (0.62 ± 0.20) | (0.43 ± 0.08) | (0.72 ± 0.14) | (0.04 ± 0.01) | (0.13 ± 0.11) |

Table 1 summarizes the product yields of the control experiments containing 10 g/L (55.5 mM/L) glucose, 10 g/L (66.6 mM/L) L-arabinose and 10 g/L (66.6 mM/L) D-xylose. The cells grown on glucose produced the acids, acetate and butyrate, as the major products, constituting more than 90% (mol/mol) of the products formed. Butyrate was the major product under glucose utilization with a 41% (g/g) yield. Ethanol, 1,3-PDO and butanol were formed in trace amounts and this shows that the growth of C. pasteurianum in glucose results in acidogenesis with little or no solventogenesis.

Similarly, the control cultures grown on L-arabinose, produced 1,3-PDO as the major product with a conversion of 28% (g/g) and a maximum yield of 36.5% (g/g). Ethanol and 1,3-PDO constituted nearly 86% (mol/mol) of the products yielded on arabinose. Growth on D-xylose resulted in the production of acetate, 1,3-PDO and ethanol as the major products with trace amounts of butyrate and butanol being formed. The control experiments with hexose and pentoses as the sole carbon source show unique array of products, based on the substrate used.

As shown in Table 2, the fermentation of glycerol results in the production 1,3-PDO and butanol as the major products, during the biphasic production of acids and solvents. The control experiment with glycerol yielded 1,3-PDO and butanol at 20% (g/g) and 22% (g/g), respectively. The ratio of butanol/1,3-PDO is close to one, 1.1 g/g (1.125 mol/mol). The objective of this experiment is focused on determining the effect of various sugars to alter this ratio, when used as co-substrates.

Effect of Glucose on Glycerol Fermentation

The addition of glucose as a co-substrate at 10 g/L (55.5 mM/L) to 20 g/L (217.2 mM/L) of glycerol, resulted in an increased production of butanol along with a simultaneous decrease in the formation of 1,3-PDO. The butanol production doubled from 0.22 g/g to 0.46 g/g with the addition of glucose at half the concentration of glycerol. At the same time, the 1,3-PDO yields dropped significantly, from 0.20 g/g to 0.07 g/g, a 65% reduction. The simultaneous reduction in the yield of 1,3-PDO and increase in the yield of butanol also results in altering the ratio of butanol/1,3-PDO to 6.57 g/g (6.33 mol/mol). This 6 fold increase in the ratio of butanol/1,3-PDO, during the addition of glucose for co-fermentation with glycerol, indicates a simultaneous consumption of the two carbon sources. The cultures grown on glucose produced only acids (acetate and butyrate) and no butanol, while cultures on glycerol produced trace amount of acids, and, butanol and 1,3-PDO as the major products. This drastic change in the distribution of products indicates a simultaneous utilization of glucose and glycerol during co-fermentation.

TABLE 2
Co-fermentation of Glycerol and Sugars: Product yields g/g (mol/mol).

| | Acetate | 1,3-PDO | Ethanol | Butyrate | Butanol |
|---|---|---|---|---|---|
| 20 g/L | 0.04 ± 0.001 | 0.20 ± 0.01 | 0.02 ± 0.002 | 0.05 ± 0.003 | 0.22 ± 0.02 |
| Glycerol | (0.06 ± 0.002) | (0.24 ± 0.01) | (0.05 ± 0.005) | (0.05 ± 0.003) | (0.27 ± 0.02) |
| 20 g/L | 0.07 ± 0.003 | 0.07 ± 0.008 | 0.03 ± 0.001 | 0.06 ± 0.005 | 0.46 ± 0.022 |
| Glycerol + | (0.11 ± 0.004) | (0.09 ± 0.01) | (0.06 ± 0.001) | (0.06 ± 0.006) | (0.57 ± 0.027) |
| 10 g/L | | | | | |
| Glucose | | | | | |
| 20 g/L | 0.05 ± 0.002 | 0.33 ± 0.03 | 0.10 ± 0.003 | 0.07 ± 0.01 | 0.21 ± 0.01 |
| Glycerol + | (0.07 ± 0.004) | (0.40 ± 0.04) | (0.20 ± 0.01) | (0.07 ± 0.01) | (0.26 ± 0.01) |
| 10 g/L | | | | | |
| L-arabinose | | | | | |
| 20 g/L | 0.03 ± 0.01 | 0.11 ± 0.02 | 0.06 ± 0.01 | 0.03 ± 0.01 | 0.19 ± 0.04 |
| Glycerol + | (0.05 ± 0.01) | (0.13 ± 0.02) | (0.13 ± 0.03) | (0.03 ± 0.01) | (0.24 ± 0.06) |
| 10 g/L | | | | | |
| D-xylose | | | | | |

The co-fermentation of glucose with glycerol using C. butyricum resulted in an increase in 1,3-PDO production. C. butyricum has the ability to produce 1,3-PDO, butyrate, acetate and ethanol from glycerol and does not possess an inherent ability to produce butanol from glycerol.

In C. acetobutylicum, it has been shown that the glycerol-glucose co-fermentation brings a shift in product pattern from an acid based production on glucose to an alcohol based production in glucose-glycerol co-fermentation (Vasconcelos, Girbal, and Soucaille 1994). In C. acetobutylicum, the shift in product during glucose-glycerol co-fermentation has been correlated to the increase in the level of NADH and ATP, along with an increase in activity of NAD-dependent alcohol dehydrogenases leading to an alcohol based fermentation.

The decrease in 1,3-PDO production in C. pasteurianum during glucose supplementation of glycerol fermentation can be explained by the presence of a possible repression of the 1,3-PDO pathway either by the presence of an alternate reduction pathway from glucose and/or catabolite repression of the glycerol dehydratase enzyme catalyzing the conversion of glycerol into 1,3-PDO through 3-hydroxypropionaldehyde. During glycerol-glucose co-metabolism, a repression in the 1,3-PDO pathway along with an increase in the activity of the enzymes in the butanol pathway (NAD-dependent butanol dehydrogenase) and an increase in the level of NAD and ATP (due to the presence of glucose and compensating for the repression in the 1,3-PDO pathway) can explain the shift in product pattern towards higher butanol formation.

The ability of C. pasteurianum to divert glycerol to produce more butanol by shutting down 1,3-PDO formation during co-fermentation with glucose clearly suggests the presence of an alternate reducing pathway to compensate for the regeneration of NAD. The availability of NAD is a key requirement to run the oxidative branch of glycerol catabolism leading to the formation of butanol. Also, from the control experiments (glucose as the sole carbon source and glycerol as the sole carbon source) and the experiments involving co-fermentation of glycerol and glucose, it is evident that the butanol production results only from the fermentation of glycerol.

Comparison of the overall product yield (total concentration of products formed/concentration of glycerol) with respect to glycerol shows that overall product yield increases from 0.53±0.025 g/g (0.68±0.032 mol/mol) to 0.69±0.022 g/g (0.89±0.028). This 30% increase in the overall product yield for glycerol during co-fermentation with glucose indicates that the supplementation of glucose not only provided an alternate reducing pathway, but also served as an energy source for cell multiplication and growth, while more of glycerol was used for solventogenesis.

Effect of Pentoses on Glycerol Fermentation

The effect of pentoses on co-metabolism with glycerol was studied using L-arabinose and D-xylose, the major products from the hydrolysis of hemicelluloses. The effect of pentoses was studied by maintaining the initial ratio of glycerol/pentose to 2:1. Control experiments were carried out with individual carbon sources at the same concentration as used in co-fermentation.

From the production data in Table 1, addition of L-arabinose to glycerol does not affect the yield of butanol. The yield (calculated with respect to glycerol) of 1,3-PDO increased by 65% to 0.33 g/g and hence the ration of butanol/1,3-PDO shifted from 1 to 0.64. The yield of ethanol also increased 5 fold from 0.02 g/g to 0.10 g/g. The solvent production by C. pasteurianum on L-arabinose predominantly resulted in the formation of 1,3-PDO and ethanol (85% of overall products) and traces of acetate, butyrate and butanol. The addition of L-arabinose did not result in an increase in the production of one compound at the expense of the other as observed in the co-fermentation of glycerol with glucose. The product formation during the co-fermentation of glycerol and L-arabinose resulted in the production of products that were observed in both the control experiments containing glycerol and L-arabinose as the sole carbon source. These results indicate that there was no product based suppression of 1,3-PDO production, which was produced from both carbon sources during co-fermentation. The addition of L-arabinose during glycerol fermentation did not alter the carbon flow of glycerol and both carbon sources were consumed in a manner independent of each other.

The addition of D-xylose to glycerol fermentation by C. pasteurianum was studied at the same concentration of 2:1. Unlike the addition of glucose and L-arabinose for co-fermentation with glycerol, which were completely consumed, addition of D-xylose resulted only in a utilization of only 25±16% of D-xylose. D-xylose was, however, effectively consumed as a sole carbon source and resulted in the production of acetate, 1,3-PDO and ethanol (88% of the overall products) with 0.78 g/g of product formation. Addition of xylose in Klebsiella pneumonia, during glycerol fermentation, resulted in an increase in the production of 1,3-PDO. In C. pasteurianum, the addition of xylose as a co-substrate during glycerol fermentation did not result in an increase in the formation of any of the products. Also the addition of xylose lowered the growth rate of the cells (FIGS. 4A and 4B).

Effect of Co-Product on Biotinaddition

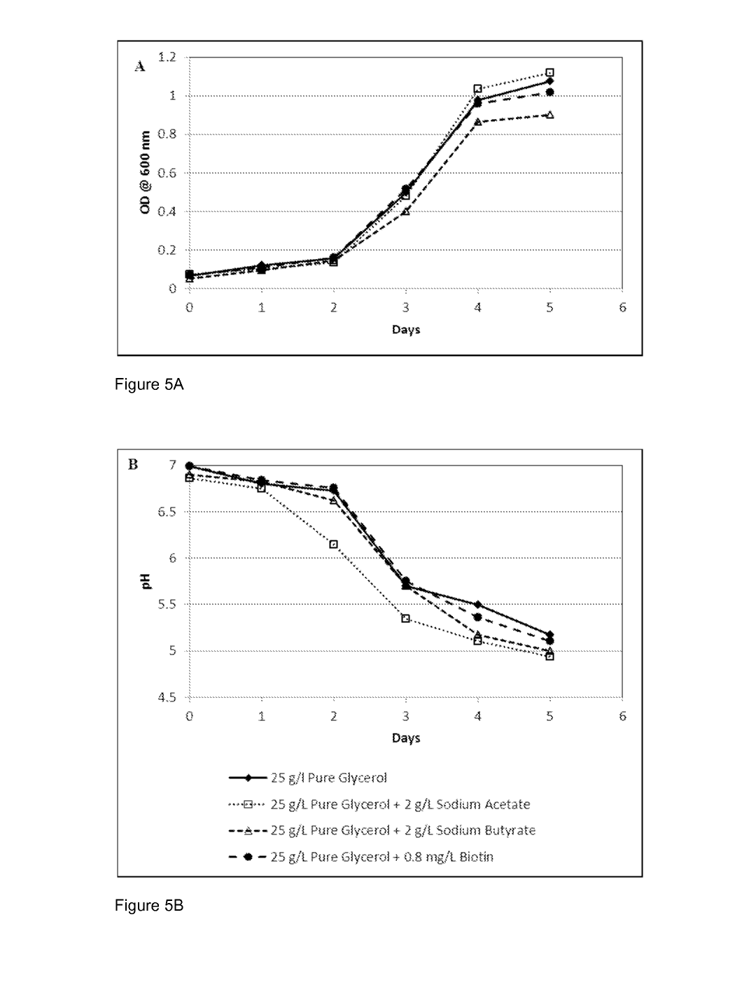

The effect of co-product addition was studied for the addition of acetate and butyrate in the form of sodium salts at 2 g/L, while the effect of biotin supplementation was studied at 0.8 mg/L. FIGS. 5A and 5B compare the growth and pH profile of the cultures on acetate and butyrate addition and biotin supplementation. Table 2 compares the product yields when co-products were added. The metabolism of the cells in terms of growth and pH was not drastically different due to the addition of compounds such as butyrate and biotin, with respect to the control (FIGS. 5A and 5B). Acetate addition resulted in a faster drop in pH and resulted in a small increase in the maximum optical density. Butyrate addition resulted in a slightly lower maximum optical density.

The product distribution during the addition of acetate resulted in a 23% increase in butanol formation (Table 3) with a corresponding decrease in 1,3-PDO production (33%). This shift of the butanol/1,3-PDO ratio due to the addition of acetate, shows the effect of acetate addition in moderating the carbon flow between the oxidation and the reduction pathway. In contrast, the addition of butyrate did not increase butanol production neither did butyrate affect an increase 1,3-PDO formation. On the other hand, the supplementation of biotin in the media altered the butanol/1,3-PDO ratio by both increasing butanol formation by 25% and decreasing 1,3-PDO by 50%.

TABLE 3
Comparison of Product Yield in g/g (mol/mol)

| | | Acetate | 1,3-PDO | Ethanol | Butyrate | Butanol |
|---|---|---|---|---|---|---|
| 25 | g/L | 0.03 ± 0.002 | 0.21 ± 0.01 | 0.07 ± 0.01 | 0.02 ± 0.01 | 0.22 ± 0.02 |
| Pure | | (0.04 ± 0.004) | (0.25 ± 0.02) | (0.15 ± 0.01) | (0.02 ± 0.01) | (0.28 ± 0.02) |
| Glycerol | | | | | | |
| 25 | g/L | 0.05 ± 0.01 | 0.14 ± 0.01 | 0.05 ± 0.01 | 0.03 ± 0.01 | 0.27 ± 0.004 |
| Pure | | (0.08 ± 0.009) | (0.17 ± 0.01) | (0.11 ± 0.02) | (0.03 ± 0.01) | (0.33 ± 0.005) |
| glycerol + | | | | | | |
| 2 | g/L | | | | | |
| Sodium | | | | | | |
| acetate | | | | | | |
| 25 | g/L | 0.05 ± 0.001 | 0.22 ± 0.005 | 0.01 ± 0.001 | 0.12 ± 0.001 | 0.21 ± 0.01 |
| Pure | | (0.08 ± 0.001) | (0.26 ± 0.01) | (0.02 ± 0.001) | (0.13 ± 0.001) | (0.27 ± 0.01) |
| glycerol + | | | | | | |
| 2 | g/L | | | | | |
| Sodium | | | | | | |
| butyrate | | | | | | |
| 25 | g/L | 0.03 ± 0.003 | 0.10 ± 0.01 | 0.02 ± 0.003 | 0.02 ± 0.01 | 0.26 ± 0.01 |
| Pure | | (0.04 ± 0.005) | (0.12 ± 0.02) | (0.04 ± 0.01) | (0.02 ± 0.01) | (0.32 ± 0.01) |
| glycerol + | | | | | | |
| 0.8 | mg/L | | | | | |

Biotin

The effect of acetate and butyrate addition on glycerol fermentation has been reported on C. butyricum and C. beijerinckii. Also reported is a decrease in 1,3-PDO formation during the supplementation of acetate in the media and an increase in butyrate was also observed. It should be noted that C. butyricum does not produce any butanol from glycerol, hence the decrease in 1,3-PDO resulted in a corresponding increase in butyrate. Butanol is the major product in C. pasteurianum and hence addition of acetate resulted in an increase in butanol. Increase in butanol production on acetate addition was reported in C. beijerinckii. The effect of acetate on increasing butanol production was correlated to the increase in the coenzyme-A transferase, an enzyme that plays a key role in the butanol pathway. There are further reports that the addition of acetate increased biomass, which was also observed in this study as the addition of acetate resulted in a noticeable increase in the optical density value (FIG. 5A). The effect of acetate addition resulted in a faster progress in metabolism as the pH drop was predominant, compared to the control.

Butyrate addition in C. butyricum was reported to have increased 1,3-PDO production with a corresponding decrease in butyrate production. Butyrate supplementation in the media was reported to increase butanol production in C. acetobutylicum. Also reported is the effect of butyrate addition at a concentration of 2 g/L and found that it resulted in an increase of butanol production by 1.6 folds. No such effect of butyrate was observed in this study. Addition of butyrate lowered the maximum optical density by 20% (FIGS. 5A and 5B). The reduction in biomass during butyrate addition was also reported in C. butyricum.

The effect of biotin supplementation was reported to increase solvent production and biomass in C. acetobutylicum. Similarly, the biotin supplementation in the media has resulted in an 18% increase in butanol formation with a corresponding decrease of 1,3-PDO formation by more than 50%.

### CONCLUSION

The effect of various compounds, from co-fermentation of sugars, addition of organic acids and supplementation of a vitamin, were studied on glycerol fermentation by C. pasteurianum. This study shows that the carbon flow from glycerol into various products can be regulated by using appropriate additions to the media. Co-fermentation of glycerol with glucose resulted in the maximum increase of butanol formation by 110%. Addition of acetate, in the form of sodium acetate at 2 g/L, and biotin (0.8 mg/L), also resulted in an increase in production of butanol (˜25%). Co-fermentation of glycerol with L-arabinose and D-xylose, respectively, did not have a positive impact on butanol yield. Similar results were obtained from addition of butyrate. Co-fermentation of glycerol and glucose is an interesting finding in this study, as it not only increases butanol production, but, it also mediates the increase in butanol through carbon shift from 1,3-PDO to butanol. Glucose, acetate and biotin can be used to maximize butanol production from glycerol, provided their addition to the media does not increase cost on feedstock.

Now, therefore, the following is claimed:

1. A method of facilitating the bioconversion of biodiesel-derived crude glycerol by a microorganism, comprising the steps of:
adjusting the pH of the crude glycerol sample to approximately 2.0-3.0;
precipitating fatty acids from the crude glycerol;
separating the fatty acids from the crude glycerol to form purified glycerol; and
combining the purified glycerol with microbial growth media to form a purified glycerol growth media.

2. The method of claim 1, further comprising the step of adding a secondary carbon source to the purified glycerol growth media.

3. The method of claim 2, wherein the secondary carbon source is a sugar.

4. The method of claim 3, wherein the sugar is glucose.

5. The method of claim 1, further comprising the step of culturing a microorganism capable of utilizing glucose as a carbon source in the purified glycerol growth media.

6. The method of claim 1, wherein the adjusting step comprises the step of adding an appropriate amount of strong acid to bring the pH to approximately 2.0-3.0.

7. The method of claim 1, wherein the separation step comprises centrifuging the sample.

8. The method of claim 5, wherein the microorganism is a bacterium.

9. The method of claim 8, wherein the bacteria is Clostridium pasteurianum.

10. The method of claim 5, further comprising the step of analyzing the purified glycerol growth media for the presence of biodiesel-derived crude glycerol bioconversion products.

11. The method of claim 5, further comprising the step of analyzing the cultured microorganism for the presence of biodiesel-derived crude glycerol bioconversion products.

12. The method of claim 10, wherein the biodiesel-derived crude glycerol conversion product is butanol.

13. The method of claim 11, wherein the biodiesel-derived crude glycerol conversion product is butanol.

14. A method of facilitating the bioconversion of biodiesel-derived crude glycerol by a microorganism, comprising the steps of:
converting unsaturated fatty acids to saturated fatty acids to form purified glycerol; and
combining the purified glycerol with microbial growth media to form a purified glycerol growth media.

15. The method of claim 14, further comprising the step of adding a secondary carbon source to the purified glycerol growth media.

16. The method of claim 15, wherein the secondary carbon source is a sugar.

17. The method of claim 16, wherein the sugar is glucose.

18. The method of claim 14, further comprising the step of culturing a microorganism capable of utilizing glucose as a carbon source in the purified glycerol growth media.

19. The method of claim 14, wherein the converting step comprises the step of contacting the crude glycerol sample with hydrogen gas in the presence of a catalyst.

20. The method of claim 18, wherein the microorganism is a bacterium.

21. The method of claim 20, wherein the bacteria is Clostridium pasteurianum.

22. The method of claim 18, further comprising the step of analyzing the purified glycerol growth media for the presence of biodiesel-derived crude glycerol bioconversion products.

23. The method of claim 18 further comprising the step of analyzing the cultured microorganism for the presence of biodiesel-derived crude glycerol bioconversion products.

24. The method of claim 22, wherein the biodiesel-derived crude glycerol conversion product is butanol.

25. The method of claim 23, wherein the biodiesel-derived crude glycerol conversion product is butanol.
